(19) United States
(12) Patent Application Publication
(10) Pub. No.: US 2012/0052047 A1
(43) Pub. Date: Mar. 1, 2012

(54) USE OF PROBIOTIC STRAIN GM-080 IN TREATING CARDIAC INFLAMMATION AND APOPTOSIS

(75) Inventors: Ying-Chen LU, CHIAYI CITY (TW); Feng-Ching HSIEH, PINGTUNG COUNTY (TW); Cheng-Chih TSAI, TAICHUNG CITY (TW); Chih-Yang HUANG, TAICHUNG CITY (TW); Hsueh-Fang WANG, TAICHUNG COUNTY (TW); Chien-Yu TSENG, CHANGHUA COUNTY (TW)

(73) Assignee: GenMont Biotech Incorporation, Tainan County (TW)

(21) Appl. No.: 12/947,829

(22) Filed: Nov. 17, 2010

(30) Foreign Application Priority Data
Sep. 1, 2010 (TW) 99129548

(51) Int. Cl. A61K 35/74; A61P 9/00

(52) U.S. Cl. 424/93.3; 424/93.45

(57) ABSTRACT

A use of probiotic strain GM-080 in treating cardiac inflammation and apoptosis is disclosed. The probiotic strain GM-080 such as Lactobacillus paracasei strain GM-080 (accession No. CCTCC M 204012) is utilized to produce a composition for treating cardiac inflammation and apoptosis in an effective dose.

### RELATED APPLICATIONS

The present application is based on, and claims priority from, Taiwan Application Serial Number 99129548, filed Sep. 1, 2010, the disclosure of which is hereby incorporated by reference herein in its entirety.

### FIELD OF THE INVENTION

This invention relates generally to a use of probiotic strain, and more particularly, to a use of probiotic strain GM-080 for treating cardiac inflammation and apoptosis.

### BACKGROUND OF THE INVENTION

Generally, probiotics (or probiotic bacteria) such as lactic acid bacteria (LAB) and some yeasts, are referred to live microorganisms for beneficial to gastrointestinal (GI) tract health, which are supplements or originally inhabit in the human body for beneficial to gastrointestinal (GI) tract health. As such for LAB, they are named as such because most of their members convert lactose and other sugars into lactic acid. LAB are also a genus of Gram-positive facultative anaerobic or microaerophilic bacteria, and they are widely applied in fermentation of food industry.

Many researches evidence that LAB can improve the allergy-related diseases and GI upset, for example, inflammatory bowel disease (IBD). Typically, LAB include main members of the genera Lactobacillus, Leuconostoc, Pediococcus, Lactococcus and Streptococcus, and other members of the genera Aerococcus, Carnobacterium, Enterococcus, Oenococcus, Sporolactobacillus, Teragenococcus, Vagococcu and Weisella. Most LAB strains belong to the genus Lactobacillus. The aforementioned LAB strains belong to the order Lactobacillales, some strains of which can serve as probiotics. In current studies, two principal kinds of probiotic bacteria, members of the genera Lactobaccilus and Bifidobacterium, have been studied in detail.

According to recent epidemiological studies, LAB can stimulate the immune response, so as to promote the immune tolerance to innocent allergens. (See Penders, J., Stobberingh, E. E., van den Brandt, P. A., Thijs, C. “The role of the intestinal microbiota in the development of atopic disorders.” European Journal of Allergy and Clinical Immunology 62(11), 1223-1236. (2007))

LAB have been evaluated in other research studies in animals and humans with respect to antibiotic-associated diarrhea, travellers' diarrhea, pediatric diarrhea, inflammatory bowel disease, irritable bowel syndrome (See Furrie, E. Probiotics and allergy. Proceedings of the Nutrition Society 64(4), 465-469, 2005; Goossens, D., Jonkers, D., Stobberingh, E., van den Bogaard, A., Russel, M., Stockbrugger, R. Probiotics in gastroenterology: indication and future perspectives. Scandinavian Journal of Gastroenterology 239 (Suppl.), 15-23, 2003; Kalliomaki, M., Salminen, S., Poussa, T., Arvilommi, H., Isolauri, E. Probiotics and prevention of atopic disease: 4-year follow-up of a randomised placebo-controlled trial. Lancet 361, 1869-1871, 2003; Pfruender, H., Amidjojo, M., Hang, F., Weuster-Botz, D. Production of Lactobacillus kefir cells for asymmetric synthesis of a 3,5-dihydroxycarboxylate. Applied Microbiology and Biotechnology 67, 619-622, 2005; Shanahan, F. Probiotics and inflammatory bowel disease: is there a scientific rationale? Inflammatory Bowel Disease 6(2), 107-115, 2000), atopic disease and so on. (See Penders, J., Stobberingh, E. E., van den Brandt, P. A., Thijs, C. The role of the intestinal microbiota in the development of atopic disorders. European Journal of Allergy and Clinical Immunology 62(11), 1223-1236, 2007; Lee, J., Seto, D., Bielory, L. Meta-analysis of clinical trials of probiotics for prevention and treatment of pediatric atopic dermatitis. Journal of Allergy and Clinical Immunology 123(1), 266-267, 2009.)

The treatment of cardiovascular disease with LAB was actually suggested more than 50 years ago when it was reported that consumption of LAB decreased blood pressure and hypocholesterolemia in humans. (See Pfruender et al., 2005; Lye, H. S., Kuan, C. Y., Ewe, J. A., Ring, W. Y., Liong, M. T. The improvement of hypertension by probiotics: Effects on cholesterol, diabetes, renin, and phytoestrogens International Journal of Molecular Sciences 10, 3755-3775, 2009).

However, prior studies are little or irrelevant to whether the probiotics can prevent cardiac inflammation and apoptosis in allergy-prone animals, so that they fail to discuss the potential mechanism of the probiotics for preventing the cardiac inflammation and apoptosis.

Therefore, it is necessary to provide a new use of the probiotic strain for treating cardiac inflammation and apoptosis, thereby developing other applications of the probiotic strains.

### SUMMARY OF THE INVENTION

Accordingly, an aspect of the present invention provides a composition for treating cardiac inflammation and apoptosis is disclosed, which may include a therapeutically effective amount of a probiotic strain GM-080 of Lactobacillus paracasei strain GM-080. L. paracasei strain GM-080 has been deposited with the China Center for Type Culture Collection (CCTCC), Wuhan University, Wuhan 430072, People's Republic of China under accession number of CCTCC M 204012 on Feb. 19, 2004.

Another aspect of the present invention provides a method for treating cardiac inflammation and apoptosis by administrating a therapeutically effective amount of probiotic strain GM-080 of Lactobacillus paracasei strain GM-080 (deposited with CCTCC of Wuhan University in China under accession No.: CCTCC M 204012), in which the probiotic strain GM-080 specifically inhibits gene expression of p-JNK, Bad and Bax.

According to the aforementioned aspect of the present invention, a composition for treating cardiac inflammation and apoptosis is disclosed. In an embodiment, the composition may include a therapeutically effective amount of a probiotic strain GM-080 of Lactobacillus paracasei strain GM-080 (deposited with CCTCC of Wuhan University in China under accession No.: CCTCC M 204012).

In an embodiment, the probiotic strain GM-080 may be live or inactivate.

In another embodiment, the probiotic strain GM-080 specifically inhibits gene expression of p-JNK, Bad and Bax.

In a further embodiment, the probiotic strain GM-080 may be a medical composition, a food additive, a food or its ingredient.

According to other aspects of the present invention, a method for treating cardiac inflammation and apoptosis by administrating a therapeutically effective amount of probiotic strain GM-080 of Lactobacillus paracasei strain GM-080 (deposited with CCTCC of Wuhan University in China under accession No.: CCTCC M 204012) is disclosed, in which the probiotic strain GM-080 specifically inhibits gene expression of p-JNK, Bad and Bax.

In an embodiment, the probiotic strain GM-080 may further include an another mixed strain.

With application to the aforementioned probiotic strain GM-080 for treating cardiac inflammation and apoptosis, the probiotic strain GM-080 can specifically inhibits gene expression of p-JNK, Bad and Bax, so as to effectively treat cardiac inflammation and apoptosis, thereby developing other applications of the probiotic strains.

### BRIEF DESCRIPTION OF THE DRAWINGS

The foregoing aspects and many of the attendant advantages of this invention are more readily appreciated as the same become better understood by reference to the following detailed description, when taken in conjunction with the accompanying drawing, wherein:

FIG. 1 depicts a diagram of signal transduction pathways involved in treatment of the cardiac inflammation and apoptosis according to an embodiment of the present invention.

FIG. 2 shows a Western blotting analysis of mouse heart tissue according to an embodiment of the present invention.

FIG. 3 depicts a bar diagram of the relative expression of p-JNK normalized to α-tubulin expression of FIG. 2.

FIG. 4 shows a bar diagram of the relative expression of JNK ½ normalized to α-tubulin expression of FIG. 2.

FIG. 5 shows a Western blotting analysis of mouse heart tissue according to another embodiment of the present invention.

FIG. 6 depicts a bar diagram of the relative expression of p-NFκB normalized to α-tubulin expression of FIG. 5.

FIG. 7 depicts a bar diagram of the relative expression of p-IκB normalized to α-tubulin expression of FIG. 5.

FIG. 8 shows a Western blotting analysis of mouse heart tissue according to a further embodiment of the present invention.

FIG. 9 depicts a bar diagram of the relative expression of Bad normalized to α-tubulin expression of FIG. 8.

FIG. 10 depicts a bar diagram of the relative expression of Bax normalized to α-tubulin expression of FIG. 8.

FIG. 11 shows a Western blotting analysis of mouse heart tissue according to a still another embodiment of the present invention.

FIG. 12 depicts a bar diagram of the relative expression of Bad normalized to α-tubulin expression of FIG. 11.

FIG. 13 depicts a bar diagram of the relative expression of caspase 3 normalized to α-tubulin expression of FIG. 11.

### DETAILED DESCRIPTION OF THE PREFERRED EMBODIMENT

Accordingly, the present invention provides a use of probiotic strain GM-080 of Lactobacillus paracasei strain GM-080 with a therapeutically effective amount for treating cardiac inflammation and apoptosis.

The term “probiotic strain GM-080” described herein refers to Lactobacillus paracasei strain GM-080. L. paracasei strain GM-080 has been deposited with the China Center for Type Culture Collection (CCTCC), Wuhan University, Wuhan 430072, People's Republic of China under accession number of CCTCC M 204012 on Feb. 19, 2004 under the Budapest Treaty and has CCTCC M 204012 as an internal Patent Deposit Designation and GM-080 as a Depositor identification Reference. The artisan in this art is familiar with the probiotic strain GM-080 obtained by the prior methods or the method of the U.S. Pat. No. 6,994,848 B2 rather than being recited in detail herein. In brief, the probiotic strain GM-080 can be cultured in MRS broth medium (DIFCO®0881) (final pH 6.5±0.2) at 37° C. under an anaerobic or aerobic condition. In another example, the MRS broth of the probiotic strain GM-080 culture may be streaked onto an agar plate.

In an embodiment, the probiotic strain GM-080 of the present invention can specifically inhibits gene expression of p-JNK, Bad and Bax, which is indicated by a series of in vivo animal immune experiments, thereby treating cardiac inflammation and apoptosis. Cardiac inflammation and apoptosis may induce myocarditis and cardiomyopathies, for example, hypersensitivity myocarditis, rheumatic heart disease (or valvular heart disease), hypertrophic cardiomyopathy, dilated cardiomyopathy, restrictive cardiomyopathy and so on. More specifically, the probiotic strain GM-080 can have a therapeutically effective amount for treating cardiac inflammation and apoptosis induced by allergic reaction. In addition, the term “animal immune experiments” described herein refers to employ “allergy-prone animal” testing model to evaluate the effect of the probiotic strain GM-080 for preventing the cardiac inflammation and apoptosis, in which those animals are artificially induced by using allergens such as ovalbumin (OVA) to cause allergy.

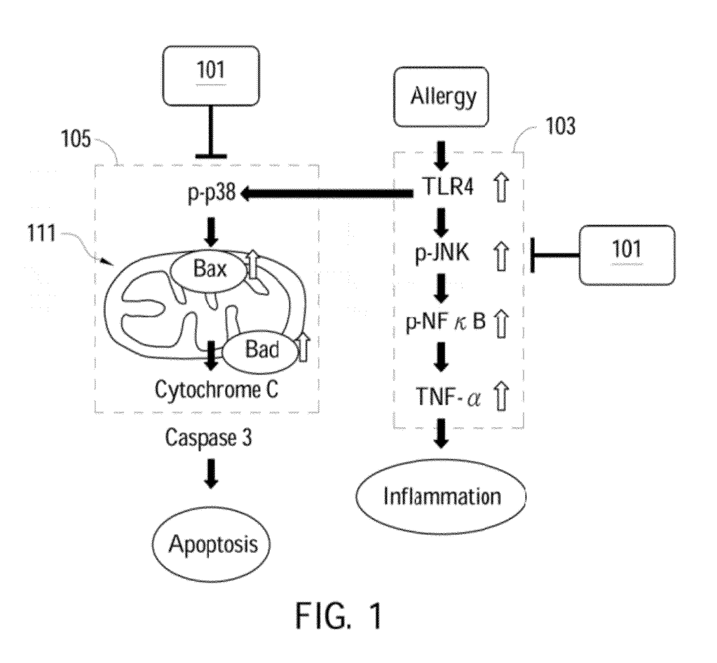

Reference is made to FIG. 1, which depicts a diagram of signal transduction pathways involved in treatment of the cardiac inflammation and apoptosis according to an embodiment of the present invention. In an embodiment, the right-sided signal transduction pathway 103 shown in FIG. 1 indicates that the probiotic strain GM-080 specifically inhibits gene expression of p-JNK, so as to suppress the downstream gene expression of p-NFκB and TNF-α, thereby eliminating the cardiac inflammation, for example, allergy-induced myocardial inflammation.

In another embodiment, the probiotic strain GM-080 also specifically inhibits gene expression of Bad and Bax to effectively prevent the Bcl-2-associated death promoter (Bad) protein and Bcl-2-associated X protein (Bax) protein from being accumulated on the mitochodrial surface, so as to suppress the downstream gene expression of cytochrome C and caspase 3, thereby eliminating the mitochondrion 111-controlled apoptosis (or programmed cell death, for example, allergy-induced myocardial apoptosis), as shown as the left-sided signal transduction pathway 105 in FIG. 1.

In a further embodiment, the probiotic strain GM-080 optionally include an another mixed strain so as to produce a composition for treating cardiac inflammation and apoptosis. In an example, the another mixed strain may include but not be limited in Lactobacillus acidophilus, Lactobacillus plantarum, Bifidobacterium longum, Lactobacillus fermentum, Lactobacillus bulgaricus, Streptococcus thermophilus, Lactobacillus cremors, Lactobacillus paracasei subsp. paracasei, Lactobacillus rhamnosus GG or any combination thereof.

It should be supplemented that the probiotic strain GM-080 (for example, Lactobacillus paracasei strain GM-080; accession No. CCTCC M 204012) may be live or inactive when it is applied in the composition for treating cardiac inflammation and apoptosis. In an example, the probiotic strain GM-080 may be a medical composition, a food additive, a food or its ingredient. In another example, the probiotic strain GM-080 may be lyophilized, and the probiotic strain GM-080 may further include other ingredients, for example, glucose, maltodextrin, baby milk, fructo-oligosaccharides, magnesium stearate, yogurt spices, other uncertain remains unseparated therefrom or any combination thereof.

Thereinafter, various applications of the probiotic strain GM-080 will be described in more details referring to several exemplary embodiments below, while not intended to be limiting. Thus, one skilled in the art can easily ascertain the essential characteristics of the present invention and, without departing from the spirit and scope thereof, can make various changes and modifications of the invention to adapt it to various usages and conditions.

### Example 1

### Establishment of Allergy-Prone Animal Testing Model

1. Preparation of Probiotic Strain GM-080

In this EXAMPLE, the probiotic strain GM-080, Lactobacillus paracasei strain GM-080 (accession No. CCTCC M 204012) may be used in animal immune experiments, so as to evaluate the effect of the probiotic strain GM-080 for preventing the cardiac inflammation and apoptosis. L. paracasei strain GM-080 (accession No. CCTCC M 204012) may have a dosage of 1×10⁶ to 1×10¹¹ CFU/g (colony-forming units per gram). L. paracasei strain GM-080 may be lyophilized, and L. paracasei strain GM-080 may further include other ingredients, for example, glucose, maltodextrin, baby milk, fructo-oligosaccharides, magnesium stearate, yogurt spices, other uncertain remains unseparated therefrom or any combination thereof.

In this embodiment, another mixed strain may be optionally added into L. paracasei strain GM-080. The appropriate mixed strain may include but not be limited in Lactobacillus acidophilus, Lactobacillus plantarum, Bifidobacterium longum, Lactobacillus fermentum, Lactobacillus bulgaricus, Streptococcus thermophilus, Lactobacillus cremors, Lactobacillus paracasei subsp. paracasei, Lactobacillus rhamnosus GG or any combination thereof. Besides, the commercially available probiotic products that contain the aforementioned mixed strains may be also used herein.

In an example, the another mixed strain may further include a first mixed strain (or a mixed strain A), in which the first mixed strain may include but not be limited in Lactobacillus acidophilus, Lactobacillus plantarum, Bifidobacterium longum, Lactobacillus fermentum, Lactobacillus bulgaricus, Streptococcus thermophilus, Lactobacillus cremors or any combination thereof. Besides, the first mixed strain may have a dosage of 1×10⁷ CFU/g or more.

In another example, the another mixed strain may further include a second mixed strain (or a mixed strain B), in which the second mixed strain may include but not be limited in Lactobacillus paracasei subsp. paracasei, Lactobacillus rhamnosus GG or any combination thereof. Besides, the second mixed strain may have a dosage of 1×10⁷ CFU/g or more.

2. Establishment of Allergy-Prone Animal Testing Model

In this EXAMPLE, BALB/c mice (purchased from BioLASCO Taiwan Co., Ltd.) are used to establish allergy-prone animal testing model. At first, all mice are randomly divided into three test groups (allergy groups) and two control groups (normal control group and allergy control group), each group of which has seven 5-week-old male BALB/c mice (normal control group, n=7) or eight 5-week-old male BALB/c mice (one allergy control group and three allergy groups, n=8 per group). The normal control group is given distilled water 0.2 ml/mice by oral administration (for example, orogastric intubation) once per day. The four allergy groups (one allergy control group and three allergy groups) are given an allergy control of 0.2 ml distilled water, different probiotic strains (Mixed Strain A, Mixed Strain B or L. paracasei strain GM-080 (CCTCC M 204012)) orally with 1×10⁶ to 1×10¹¹ CFU/g per mouse and once per day. The mice of the allergy control group and allergy groups are intra-peritoneally injected with 2 μg, 6 μg/mouse of OVA mixed with complete Freund's adjuvant (CFA) on days 0 and 14, respectively, so as to induce the allergy reaction of those mice. All mice are weighed and decapitated after the 28-day treatment process. The mice hearts are then excised and cleaned with the distilled water. The left, right atriums and ventricles are separated and weighed.

Ambient temperature is controlled at 25±1° C., relative humidity at 65±5%. In addition, the animals are maintained on a reverse 12 h light-dark cycle. The light period began at 7:00 a.m. Mice are provided with standard laboratory chow (MF-18; ORIENTAL YEAST CO., LTD) and water ad libitum. All experimental procedures are performed according to the NIH Guide for the Care and Use of Laboratory Animals.

3. Heart Tissue Extraction

Cardiac tissue extracts are obtained by homogenizing the left ventricle samples in a lysis buffer (20 mM Tris, 2 mM EDTA, 50 mM 2-mercaptoethanol, 10% glycerol, pH 7.4, proteinase inhibitor (Roche), phosphatase inhibitor cocktail (sigma)) at a ratio of 100 mg tissue/1 ml buffer for 1 min. The homogenates are placed on ice for 10 min., and then centrifuged at 12,000×g for 40 minutes twice. The supernatant is collected and stored at −80° C. for subsequent experiments.

### Example 2

### Evaluation of Effect of Probiotic Strain GM-080 for Preventing Cardiac Inflammation And Apoptosis

In this EXAMPLE, an electrophoresis analysis and Western blotting assay are used to evaluate the effect of the probiotic strain GM-080 for preventing the cardiac inflammation and apoptosis. The detail is described as follows.

The extracted tissue samples are prepared as described as above. Sodium dodecyl sulfate-polyacrylamide gel electrophoresis (SDS-PAGE) is performed with 10% polyacrylamide gels. The samples are then electrophoresed at 85 V for 3.5 h and equilibrated for 15 min in 25 mM Tris-HCl, pH 8.3, containing 192 mM glycine and 20% (V/V) methanol. The preparation of SDS-PAGE and related equipments are familiar with the artisan in this art of the present invention rather than being recited in detail herein.

Electrophoresed proteins are transferred to a transfer membrane such as polyvinylidene difluoride (PVDF) membrane (Millipore, Bedford, Mass., 0.45 in pore size) with a Western blotting kit, for example, a Bio-rad Scientific Instruments Transphor Unit, at 85 V for 2.5 h. And then, PVDF membranes are incubated at room temperature for 1 h in a blocking buffer containing 5% non-fat milk and a TBS buffer (iris-Base, NaCl, Tween-20, pH 7.4). First antibodies are diluted to 1:500 in an antibody-binding buffer overnight at 4° C. The immunoblots are washed three times in a TBS buffer for 10 min during each phase, and then immersed in the second antibody solution for 1 h and diluted 500-fold in the TBS buffer. The immunoblots are then washed in a blotting buffer three times for 10 min each phase. The immunoblotted proteins are visualized by using an enhanced chemiluminescence ECL Western blotting luminal Reagent (Santa Cruz, Calif., USA) and quantified using a Fujifilm LAS-3000 chemiluminescence detection system (Tokyo, Japan).

The first antibodies described as the aforementioned may include TNF-α (Cell Signaling. Technology, Beverly, Mass., USA), Toll-like receptor 4 (TLR4), phospholate-Jun-N-terminal kinase (p-JNK), phospholate-nuclear factor-KB (p-NFκB), phospholate-IκB (p-IκB), phospholate-p 38 (p-p38), Bad, Bax, cytochrome c, caspase 3 and α-tubulin (those are purchased from Santa Cruz Biotechnology, Santa Cruz, Calif., USA). The second antibody solution described as the aforementioned may contain goat anti-mouse IgG-HRP, goat anti-rabbit IgG-HRP, or donkey anti-goat IgG-HRP (those are purchased from Santa Cruz Biotechnology, Santa Cruz, Calif., USA).

The antibody binding buffer described as the aforementioned may contain 100 mM Tris-HCl, pH 7.5, 0.9% (w/v) NaCl, 0.1% (v/v) Tween-20.

In addition, each example herein and hereafter is repeated a minimum of three times, and data are expressed as mean±SD. One-way ANOVA with a Tukey-Kramer procedure for multiple comparisons is used to examine the statistical differences between treatments. Differences were considered as significant at p<0.05.

1. Evaluation of Influence of Probiotic Strain GM-080 Involving in Gene Expression of p-JNK and JNK ½ in Allergy-Prone Mouse Hearts

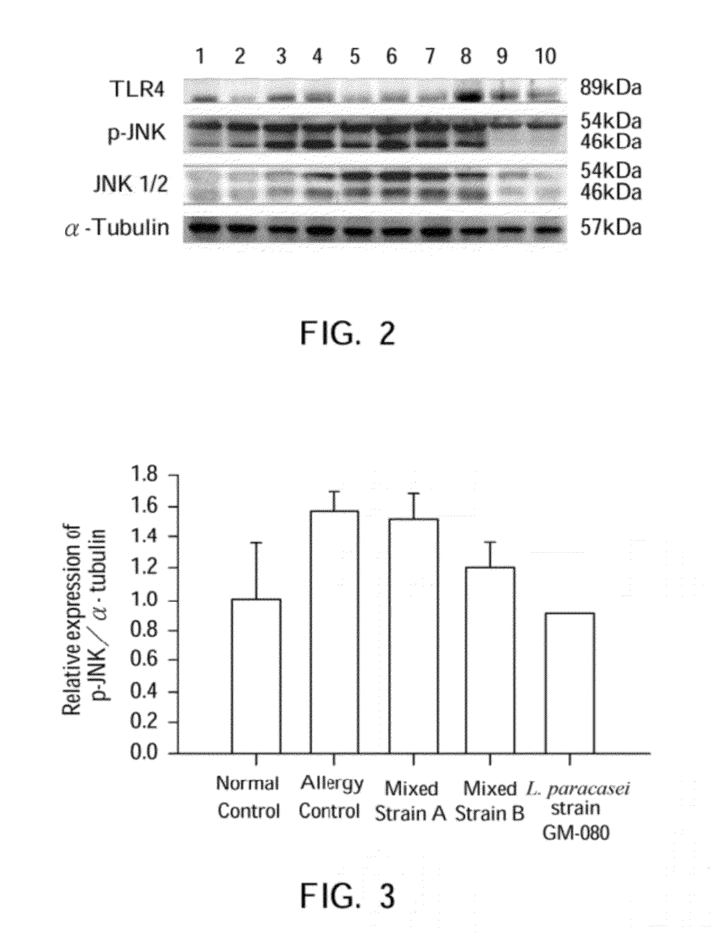

Reference is made to FIG. 2, which shows a Western blotting analysis of mouse heart tissue according to an embodiment of the present invention, in which the lines 1 to 2 refer to the normal control group, the lines 3 to 4 refer to the allergy control group, the lines 5 to 6 refer to the allergy group orally administrated with the mixed strain A (i.e. the first mixed strain), the lines 7 to 8 refer to the allergy group orally administrated with the mixed strain B (i.e. the second mixed strain), the lines 9 to 10 refer to the allergy group orally administrated with L. paracasei strain GM-080 (accession No. CCTCC M 204012). FIG. 2 shows the protein expression of TLR4 (about 89 kDa), p-JNK (about 54 kDa and about 46 kDa) and JNK ½ (about 54 kDa and about 46 kDa) in the heart tissues of allergy-prone mice. The α-tubulin (about 57 kDa) amount is detected as an internal control for normalizing those protein amounts.

Reference is made to FIG. 3, which depicts a bar diagram of the relative expression of p-JNK normalized to α-tubulin expression of FIG. 2, in which the vertical axis refers to the relative expression of p-JNK normalized to α-tubulin expression (i.e. relative expression of p-JNK/α-tubulin), and the relative expression of p-JNK/α-tubulin of the normal group is set to 1.0.

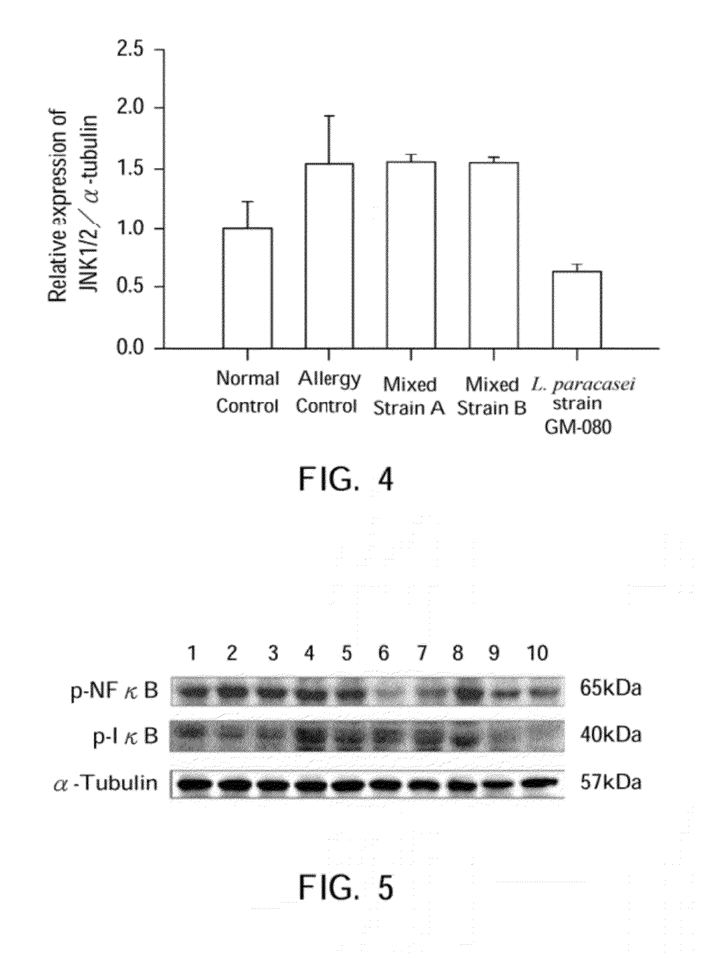

Reference is made to FIG. 4, which depicts a bar diagram of the relative expression of JNK ½ normalized to α-tubulin expression of FIG. 2, in which the vertical axis refers to the relative expression of JNK ½ normalized to α-tubulin expression (i.e. relative expression of JNK ½/α-tubulin), and the relative expression of JNK ½/α-tubulin of the normal group is set to 1.0.

According to the results of FIGS. 2 to 4, in comparison with the normal control group (the lines 1 to 2 of FIG. 2), the expression of p-JNK and JNK ½ is significantly increased in the Mouse heart tissues of the allergy control group (the lines 3 to 4 of FIG. 2) after the mice are sensitized by allergens. And in comparison with the allergy control group (the lines 3 to 4 of FIG. 2), the expression of p-JNK and JNK ½ in the mouse heart tissues of the allergy group (the lines 9 to 10 of FIG. 2, oral administration of L. paracasei strain GM-080) is significantly lower than in the allergy groups (the lines 5 to 8 of FIG. 2, oral administration of the mixed strain A or B) and the allergy control group (the lines 3 to 4 of FIG. 2) according to the results of FIGS. 3 to 4. The result indicates that the oral administration of probiotic strain GM-080 of EXAMPLE 1 specifically inhibits the gene expression of p-JNK and JNK ½ in the allergy-prone mouse hearts.

2. Evaluation of Influence of Probiotic Strain GM-080 Involving in Gene Expression of p-NFκB and p-IκB in Allergy-Prone Mouse Hearts

Reference is made to FIG. 5, which shows a Western blotting analysis of mouse heart tissue according to another embodiment of the present invention, in which the lines 1 to 2 refer to the normal control group, the lines 3 to 4 refer to the allergy control group, the lines 5 to 6 refer to the allergy group orally administrated with the mixed strain A (i.e. the first mixed strain), the lines 7 to 8 refer to the allergy group orally administrated with the mixed strain B (i.e. the second mixed strain), the lines 9 to 10 refer to the allergy group orally administrated with L. paracasei strain GM-080 (accession No. CCTCC M 204012). FIG. 5 shows the protein expression of p-NFκB (about 65 kDa) and p-IκB (about 40 kDa) in the heart tissues of allergy-prone mice. The α-tubulin (about 57 kDa) amount is detected as an internal control for normalizing those protein amounts.

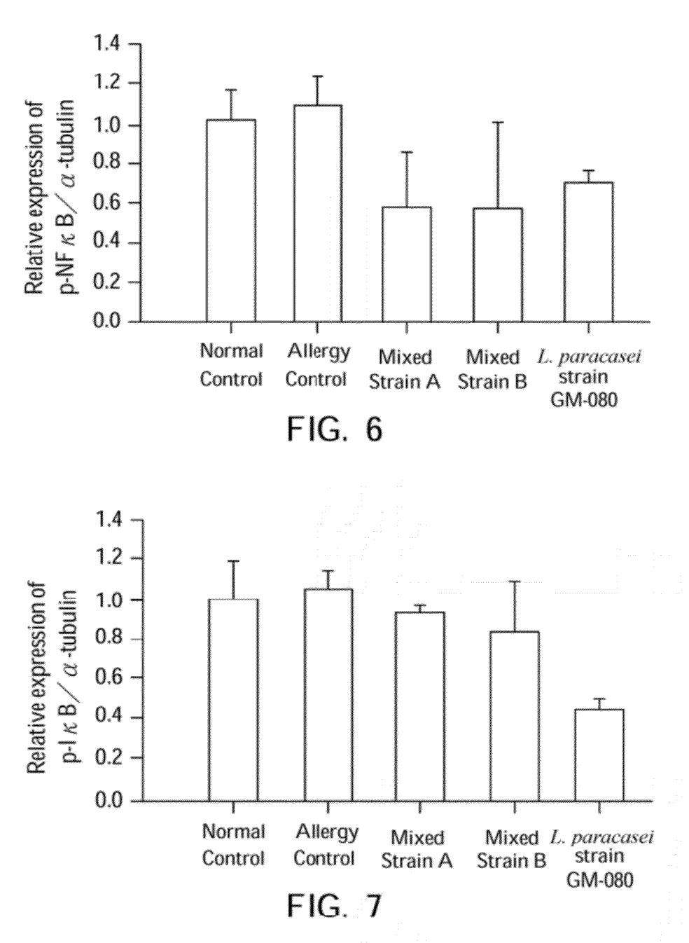

Reference is made to FIG. 6, which depicts a bar diagram of the relative expression of p-NFκB normalized to α-tubulin expression of FIG. 5, in, which the vertical axis refers to the relative expression of p-NFκB normalized to α-tubulin expression (i.e. relative expression of p-NFκB/α-tubulin), and the relative expression of p-NFκB/α-tubulin of the normal group is set to 1.0.

Reference is made to FIG. 7, which depicts a bar diagram of the relative expression of p-IκB normalized to α-tubulin expression of FIG. 5, in which the vertical axis refers to the relative expression of p-IκB normalized to α-tubulin expression (i.e. relative expression of p-IκB/α-tubulin), and the relative expression of p-IκB/α-tubulin of the normal group is set to 1.0.

According to the results of FIGS. 5 to 7, in comparison with the normal control group (the lines 1 to 2 of FIG. 5), the expression of p-NFκB and p-IκB is slightly increased in the mouse heart tissues of the allergy control group (the lines 3 to 4 of FIG. 5) after the mice are sensitized by allergens. And according to the results of FIGS. 5 to 7, in comparison with the allergy control group (the lines 3 to 4 of FIG. 5), the expression of p-NFκB in the mouse heart tissues of the allergy group (the lines 9 to 10 of FIG. 5, oral administration of L. paracasei strain GM-080) is significantly lower than in the allergy control group (the lines 3 to 4 of FIG. 5), and the expression of p-IκB in the mouse heart tissues of the allergy group (the lines 9 to 10 of FIG. 5, oral administration of L. paracasei strain GM-080) is significantly lower than in the allergy groups (the lines 5 to 8 of FIG. 5, oral administration of the mixed strain A or B) and the allergy control group (the lines 3 to 4 of FIG. 5). The result indicates that the oral administration of probiotic strain GM-080 of EXAMPLE 1 specifically inhibits the gene expression of p-NFκB and p-IκB in the allergy-prone mouse hearts.

3. Evaluation of Influence of Probiotic Strain GM-080 Involving in Gene Expression of Bad and Bax in Allergy-Prone Mouse Hearts

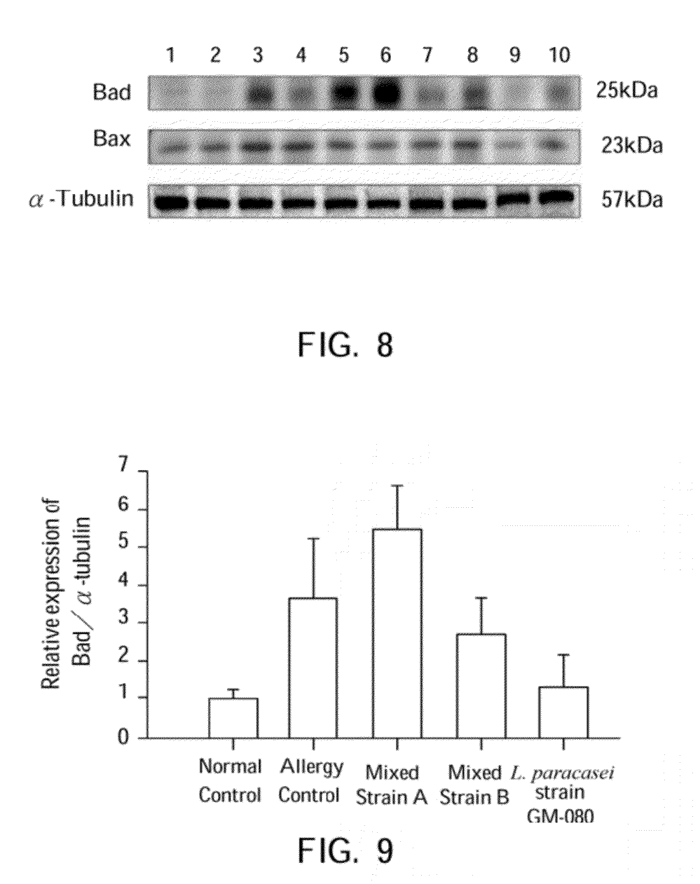

Reference is made to FIG. 8, which shows a Western blotting analysis of mouse heart tissue according to a further embodiment of the present invention, in which the lines 1 to 2 refer to the normal control group, the lines 3 to 4 refer to the allergy control group, the lines 5 to 6 refer to the allergy group orally administrated with the mixed strain A (i.e. the first mixed strain), the lines 7 to 8 refer to the allergy group orally administrated with the mixed strain B (i.e. the second mixed strain), the lines 9 to 10 refer to the allergy group orally administrated with L. paracasei strain GM-080 (accession No. CCTCC M 204012). FIG. 8 shows the protein expression of Bad (about 25 kDa) and Bax (about 23 kDa) in the heart tissues of allergy-prone mice. The α-tubulin (about 57 kDa) amount is detected as an internal control for normalizing those protein amounts.

Reference is made to FIG. 9, which depicts a bar diagram of the relative expression of Bad normalized to α-tubulin expression of FIG. 8, in which the vertical axis refers to the relative expression of Bad normalized to α-tubulin expression (i.e. relative expression of Bad/α-tubulin), and the relative expression of Bad/α-tubulin of the normal group is set to 1.0.

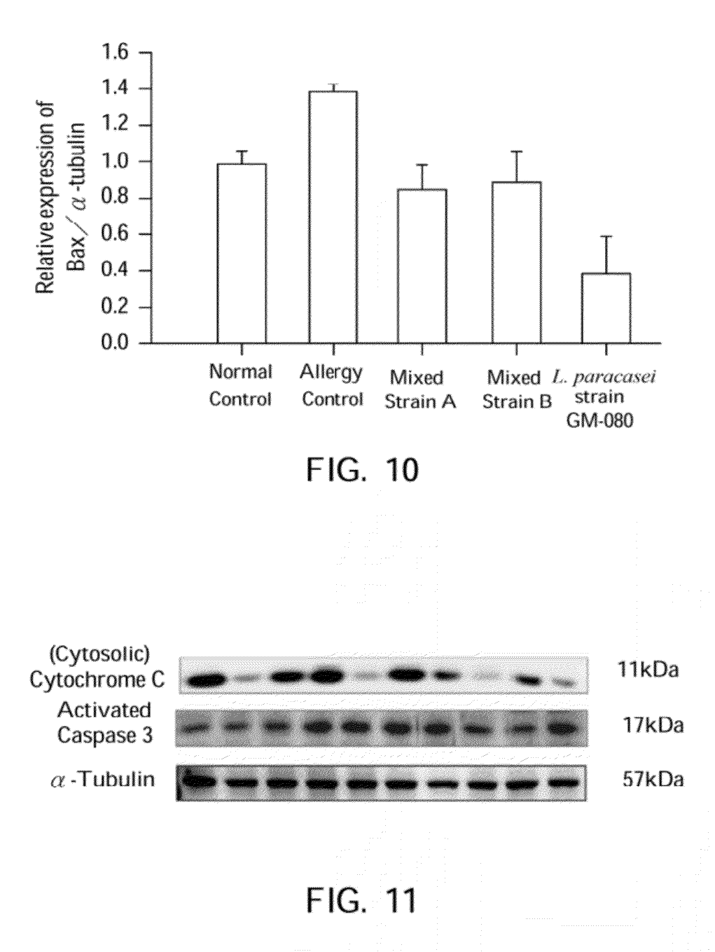

Reference is made to FIG. 10, which depicts a bar diagram of the relative expression of Bax normalized to α-tubulin expression of FIG. 8, in which the vertical axis refers to the relative expression of Bax normalized to α-tubulin expression (i.e. relative expression of Bax/α-tubulin), and the relative expression of Bax/α-tubulin of the normal group is set to 1.0.

According to the results of FIGS. 8 to 10, in comparison with the normal control group (the lines 1 to 2 of FIG. 8), the expression of Bad and Bax is significantly increased in the mouse heart tissues of the allergy control group (the lines 3 to 4 of FIG. 8) after the mice are sensitized by allergens. And according to the results of FIGS. 8 to 10, in comparison with the allergy control group (the lines 3 to 4 of FIG. 8), the expression of Bad and Bax in the mouse heart tissues of the allergy group (the lines 9 to 10 of FIG. 8, oral administration of L. paracasei strain GM-080) is significantly lower than in the allergy groups (the lines 5 to 8 of FIG. 8, oral administration of the mixed strain A or B) and the allergy control group (the lines 3 to 4 of FIG. 8). The result indicates that the oral administration of probiotic strain GM-080 of EXAMPLE 1 specifically inhibits the gene expression of Bad and Bax in the allergy-prone mouse hearts.

4. Evaluation of Influence of Probiotic Strain GM-080 Involving in Gene Expression of Cytochrome C and Caspase 3 in Allergy-Prone Mouse Hearts

Reference is made to FIG. 11, which shows a Western blotting analysis of mouse heart tissue according to a still another embodiment of the present invention, in which the lines 1 to 2 refer to the normal control group, the lines 3 to 4 refer to the allergy control group, the lines 5 to 6 refer to the allergy group orally administrated with the mixed strain A (i.e. the first mixed strain), the lines 7 to 8 refer to the allergy group orally administrated with the mixed strain B (i.e. the second mixed strain), the lines 9 to 10 refer to the allergy group orally administrated with L. paracasei strain GM-080 (accession No. CCTCC M 204012). FIG. 11 shows the protein expression of cytochrome C (about 11 kDa) and caspase 3 (about 17 kDa) in the heart tissues of allergy-prone mice. The α-tubulin (about 57 kDa) amount is detected as an internal control for normalizing those protein amounts.

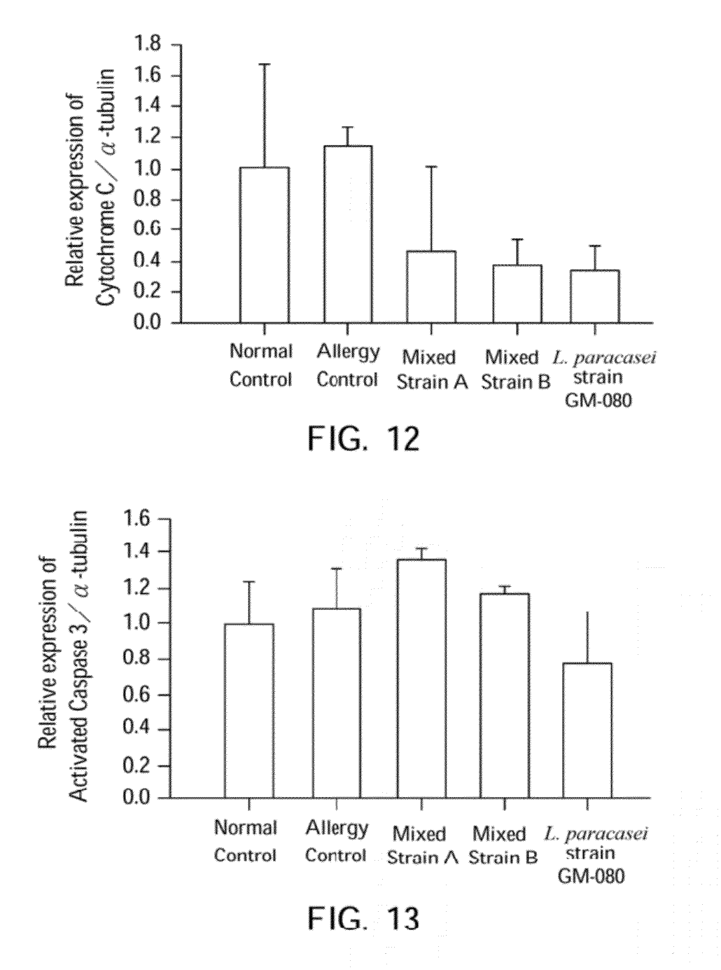

Reference is made to FIG. 12, which depicts a bar diagram of the relative expression of Bad normalized to α-tubulin expression of FIG. 11, in which the vertical axis refers to the relative expression of cytochrome C normalized to α-tubulin expression (i.e. relative expression of cytochrome C/α-tubulin), and the relative expression of cytochrome C/α-tubulin of the normal group is set to 1.0.

Reference is made to FIG. 13, which depicts a bar diagram of the relative expression of caspase 3 normalized to α-tubulin expression of FIG. 11, in which the vertical axis refers to the relative expression of caspase 3 normalized to α-tubulin expression (i.e. relative expression of caspase 3/α-tubulin), and the relative expression of caspase 3/α-tubulin of the normal group is set to 1.0.

According to the results of FIGS. 11 to 13, in comparison with the normal control group (the lines 1 to 2 of FIG. 11), the expression of cytochrome C and caspase 3 is slightly increased in the mouse heart tissues of the allergy control group (the lines 3 to 4 of FIG. 11) after the mice are sensitized by allergens. And according to the results of FIGS. 11 to 13, in comparison with the allergy control group (the lines 3 to 4 of FIG. 11), the expression of cytochrome C and caspase 3 in the mouse heart tissues of the allergy group (the lines 9 to 10 of FIG. 11, oral administration of L. paracasei strain GM-080) is significantly lower than in the allergy groups (the lines 5 to 8 of FIG. 11, oral administration of the mixed strain A or B) and the allergy control group (the lines 3 to 4 of FIG. 11). The result indicates that the oral administration of probiotic strain GM-080 of EXAMPLE 1 specifically inhibits the gene expression of cytochrome C and caspase 3 in the allergy-prone mouse hearts.

In summary, the present invention is evidenced that the probiotic strain GM-080 (L. paracasei strain GM-080: accession No. CCTCC M 204012) of the present invention can be applied in the treatment of cardiac inflammation and apoptosis, and the probiotic strain GM-080 is potentially involved in the mechanism of the specific inhibition of the gene expression of p-JNK, Bad and Bax, so as to treat cardiac inflammation and apoptosis, thereby exploiting other applications of the probiotic strains. However, it is necessarily supplemented that, specific strains, specific analysis methods, specific animal models, specific reaction conditions, specific immunization ways, specific materials or specific apparatuses are employed as exemplary embodiments for clarifying the use of the probiotic strain GM-080 for treating cardiac inflammation and apoptosis of the present invention. However, as is understood by a person skilled in the art, other strains, other analysis methods, other animal models, other reaction conditions, other immunization ways, other comparable materials or apparatuses can be also employed in the composition for treating cardiac inflammation and apoptosis of the present invention, rather than limiting to thereto. In addition, when the probiotic strain GM-080 is applied in a composition of a medical composition, a food additive, a food or its ingredient, the probiotic strain GM-080 is live, inactivate or lyophilized. Moreover, the probiotic strain GM-080 may further include other ingredients, for example, glucose, maltodextrin, baby milk, fructo-oligosaccharides, magnesium stearate, yogurt spices, other uncertain remains unseparated therefrom or any combination thereof.

According to the embodiments of the present invention, the aforementioned probiotic strain GM-080 for treating cardiac inflammation and apoptosis, the probiotic strain GM-080 advantageously and specifically inhibits gene expression of p-JNK, Bad and Bax, so as to effectively treat cardiac inflammation and apoptosis, thereby developing other applications of the probiotic strains.

As is understood by a person skilled in the art, the foregoing embodiments of the present invention are illustrated of the present invention rather than limiting of the present invention. It is intended to cover various modifications and similar arrangements included within the spirit and scope of the appended claims. Therefore, the scope of which should be accorded the broadest interpretation so as to encompass all such modifications and similar structure.

What is claimed is:

1. A composition for treating cardiac inflammation and apoptosis which comprises a therapeutically effective amount of a probiotic strain GM-080 of Lactobacillus paracasei strain GM-080 (deposited at the China Center for Type Culture Collection of Wuhan University in China under accession No.: CCTCC M 204012).

2. The composition according to claim 1, wherein the probiotic strain GM-080 is live or inactivate.

3. The composition according to claim 1, wherein probiotic strain GM-080 specifically inhibits gene expression of phosphorylated c-Jun N-terminal kinase (p-JNK).

4. The composition according to claim 1, wherein the probiotic strain GM-080 specifically inhibits gene expression of Bcl-2-associated death promoter (Bad) and Bcl-2-associated X protein (Bax).

5. The composition according to claim 1, wherein the probiotic strain GM-080 is an active ingredient with the therapeutically effective amount for treating cardiac inflammation and apoptosis.

6. The composition according to claim 1, wherein the composition is a medical composition, a food additive, a food or its ingredient.

7. A method for treating cardiac inflammation and apoptosis by administrating a therapeutically effective amount of probiotic strain GM-080 of Lactobacillus paracasei strain GM-080 (deposited at the China Center for Type Culture Collection of Wuhan University in China under accession No.: CCTCC M 204012) to specifically inhibit gene expression of p-JNK, Bad and Bax.

8. The method according to claim 7, wherein the probiotic strain GM-080 is live or inactive.

9. The method according to claim 7, wherein the probiotic strain GM-080 further comprises an another mixed strain.

10. The method according to claim 9, wherein the another mixed strain is selected from the group consisting of Lactobacillus acidophilus, Lactobacillus plantarum, Bifidobacterium longum, Lactobacillus fermentum, Lactobacillus bulgaricus, Streptococcus thermophilus, Lactobacillus cremors, Lactobacillus paracasei subsp. paracasei, Lactobacillus rhamnosus GG and any combination thereof.
